(19) United States
(12) Patent Application Publication
(10) Pub. No.: US 2007/0163587 A1
(43) Pub. Date: Jul. 19, 2007

(54) Oral respirator device and method for mask-free filtering of particulates from breathed air

(76) Inventor: Jeffrey L. Teibel, Rochester, NY (US)

Correspondence Address: HOWARD J. GREENWALD P.C., 349 W. COMMERCIAL STREET SUITE 3075, EAST ROCHESTER, NY, 14445-2408, US

(21) Appl. No.: 11/334,841

(22) Filed: Jan. 19, 2006

(51) Int. Cl. A61M 16/00; A62B 23/02

(52) U.S. Cl. 128/204.18; 128/205.27

(57) ABSTRACT

An oral respirator device and method for mask-free filtering of particulates from breathed air. A surgical mask is removably disposed within a housing. The housing has an air chamber, a first air inlet, a second air inlet, and a means of securing the surgical mask filter. Operably attached to the housing is an oral interface (such as a surgical tube). In one embodiment, the oral interface has three openings, one of which is a mouthpiece while the other two openings, the ends of the tube, are connected to the air inlets of the housing. Environmental air enters the air chamber of the housing via the filter becoming filtered breathable air. The filtered breathable air enters the mouth of the user via the oral interface. In one embodiment, one-way valves are operatively connected to the oral interface.

### FIELD OF THE INVENTION

An oral respirator device and method for mask-free filtering of particulates from breathed air comprising a filter removably disposed within said oral respirator, wherein said filter is a surgical mask, a housing for receiving and confining said filter comprising an air chamber, a first air inlet, a second air inlet, and a means of securing said filter, an oral interface comprising a tubular member operatively connected to said housing, wherein said tubular member comprises a first opening, a second opening, and a third opening, a first valve operatively connected to said tubular member, a second valve operatively connected to said tubular member, wherein said oral interface enables a user to breathe through said respirator, and wherein said oral respirator filters particulates from breathed air.

### BACKGROUND OF THE INVENTION

In environments having noxious particulates suspended in the air, filtration devices are commonly employed to filter the air inhaled by the user. Since people frequently breathe through both the nasal passages and the mouth while inhaling, filtering of the particulates from the environmental air is typically accomplished with a face mask that fits over the mouth and nose area of the face with ties for securing the mask around the back of the head.

In particular, there is a need to filter environmental air into breathable air during various medical procedures, thereby preventing particulates potentially carrying bacteria, fungi and the like from entering the mouth or nasal passages of the person performing the medical procedure. For example, when a podiatrist performs certain procedures on the feet and toenails, these procedures often generate particulates that may be carriers of infectious organisms. Similarly, a need arises for nail technicians during manicures, pedicures, “fill-ins,” or application of faux nails. A similar need to filter fine particulates from the air might arise in any number of contexts including emergency workers or victims in disaster scenes with smog, fire, smoke, dust, debris or the like suspended in the air. Other examples in which the present invention may be advantageously used would include industrial, construction, mining, excavation worksites, and the like.

Other prior art respiration devices are relatively complex, often involving valves, power supplies, motors, fans and electronic parts that render them costly to manufacture and maintain. They may also require specialized filters that are costly and not readily available through established supply channels in the medical and industrial community. The present invention offers significant advantages in its simplicity and absence of complex mechanical or electrical parts. To increase the comfort of the user, the present invention additionally offers advantages in the way of a portable, compact, lightweight design.

Surgical-type masks typically worn by medical professionals are relatively effective as filtering systems, however, they have many drawbacks. Most importantly, users often find the masks uncomfortable. They may have insufficient venting and cause excess heat build-up, cause fogging if the user wears eyeglasses or magnifying devices, cause bothersome reflections, cause sweating, irritate the skin, tangle in the hair, and interfere with eyeglasses or other medical and nonmedical equipment that is affixed to the head and neck. These face masks inhibit the ability of the medical professional to freely communicate with the patient, especially with hearing impaired patients who may rely partially or entirely on visual cue perception (e.g. lip reading) for communication.

It is therefore, an object of the present invention to provide an oral device for filtering environmental or breathed air for preventing a user from breathing unfiltered air.

It is another object of the present invention to provide a portable mask-free oral respirator device that is durable and reliable.

It is yet another object of this invention to provide a device that provides an oral respirator option for the user.

It is yet another object of this invention to provide a relatively simple oral respirator device that is economical from the viewpoint of the manufacturer and consumer.

It is yet another object of the present invention to provide a mask-free respirator device and method for filtering breathable air that is susceptible of low manufacturing costs with regard to labor and materials, and which accordingly is then susceptible of low prices for the consuming public, thereby making it economically available to the buying public.

It is yet another object of this invention to provide a relatively simple respirator device that contains no batteries or electronic components such that it is both durable and operable without an external power supply.

It is yet another object of this invention to provide a device that incorporates a low-cost filter that is well known, almost ubiquitous in the medical and industrial world, and available through long-established supply channels.

It is yet another object of this invention to provide a device that does not require specialized filters and require additional inventory supplies for medical practitioners and industrial users by utilizing prior art and commercially available filters.

It is yet another object of this invention to provide a respirator device that filters particulates from the air and renders the user's breathable air safe for inhalation under applicable safety and health regulations.

### SUMMARY OF THE INVENTION

In accordance with this invention, and in one embodiment thereof, there is provided an oral respirator device and method for mask-free filtering of particulates from breathed air comprising a filter removably disposed within said oral respirator, wherein said filter is a surgical mask, a housing for receiving and confining said filter comprising an air chamber, a first air inlet, a second air inlet, and a means of securing said filter, an oral interface comprising a tubular member operatively connected to said housing, wherein said tubular member comprises a first opening, a second opening, and a third opening, a first valve operatively connected to said tubular member, a second valve operatively connected to said tubular member, wherein said oral interface enables a user to breathe through said respirator, and wherein said oral respirator filters particulates from breathed air.

Thus, having broadly outlined the more important features of the present invention in order that the detailed description thereof may be better understood, and that the present contribution to the art may be better appreciated, there are, of course, additional features of the present invention that will be described herein and will form a part of the subject matter of the claims appended to this specification.

In this respect, before explaining at least one embodiment of the invention in detail, it is to be understood that the invention is not limited in its application to the details of construction and the arrangements of the components set forth in the following description or illustrated in the drawings. The present invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent construction insofar as they do not depart from the spirit and scope of the conception regarded as the present invention.

### BRIEF DESCRIPTION OF THE DRAWINGS

The invention will be described by reference to the specification and the drawings, in which like numerals refer to like elements, and wherein:

FIG. 1 depicts a preferred embodiment of an oral respirator device for mask-free filtering of breathed air;

FIG. 2A depicts a preferred embodiment of the housing of FIG. 1;

FIG. 2B depicts another view of the preferred embodiment of the housing shown in FIG. 2A;

FIG. 3 depicts an alternate embodiment of an oral respirator;

FIG. 4 depicts one portion of the housing depicted in FIG. 3;

FIG. 5 depicts an alternate embodiment of one portion of the housing;

FIG. 6 depicts one embodiment of both portions of the housing and how such portions are mated together while in use;

FIG. 7 depicts another view of the embodiment of both portions of the housing in FIG. 6 and how such portions are mated together while in use;

FIG. 8 depicts an alternate embodiment of the housing of the device in FIG. 3;

FIG. 9 depicts another embodiment of both portions of the housing and how such portions are mated together while in use;

FIG. 10 depicts an alternate embodiment of the housing in FIG. 9;

FIG. 11 depicts an alternate embodiment of the device in FIG. 3;

FIG. 12 depicts a detailed view of a portion of the housing and the oral interface depicted in FIG. 3 and how such parts are operably connected;

FIG. 13A depicts a one-way airflow bifurcated oral interface;

FIG. 13B depicts a two-way airflow bifurcated oral interface; and

FIGS. 14A, 14B, 14C, 14D and 15 depict a user using the oral respirator device for mask-free filtering of breathed air.

### DETAILED DESCRIPTION OF A PREFERRED EMBODIMENT

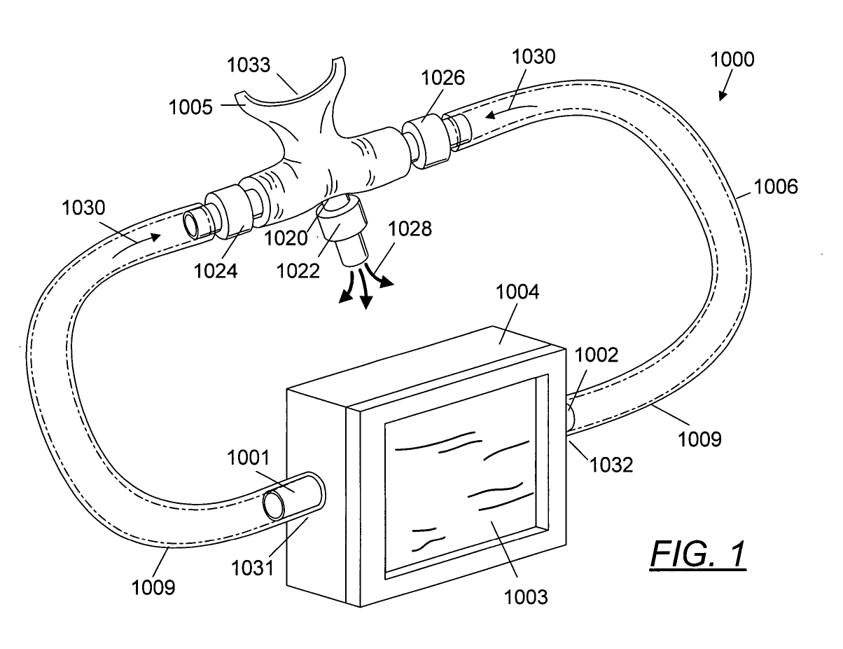

FIG. 1 depicts one embodiment of an oral respirator device 1000 for mask-free filtering of breathed air. Specifically, it will be noted in the various figures that the device relates to a mask-free respirator for filtering particulates from breathed air. In its broadest context, referring to FIG. 1 and the embodiment depicted therein, the present invention consists of a housing 1004, an oral interface 1006 and a filter 1003 arranged directly upstream in a direction of airflow 1030. Such components are individually configured and correlated with respect to each other so as to attain the desired objective, to wit, a filtration device capable of removing a sufficient amount of contaminants from the air to render it safely breathable to the user. As used is this specification, safe means complying with applicable statutes and regulations concerning health or air quality. By way of example, certifications and regulations may be issued by authorities and organizations such as OSHA, CDC, and CE. More particularly, exemplary regulations include EC Directive 89/686 and EN149:2001 FFP2D.

Referring again to the embodiment depicted in FIG. 1, oral respirator device 1000 comprises a filter 1003 removably disposed within said oral respirator 1000. In one embodiment, said filter 1003 comprises a surgical mask. Use of a common surgical mask as a filter 1003 is one of the advantageous features of the present invention. The incorporation of a surgical mask eliminates the necessity of specialized filters and allows the user to incorporate items commonly available in a hospital, clinic or medical office.

As used in this specification securing assembly for a filter shall mean any structure that removably affixes a filter within a housing. By way of example, several structures are disclosed and described herein. More particularly, reference is made to FIGS. 1, 2A, 2B, 3, 4, 6, 7, 9, 10 and 11 and the accompanying disclosure.

As used in this specification, the term surgical mask shall mean surgical face masks and half masks that shield the nose, mouth and chin are of the user as well as equivalent devices such as filtration masks, bacterial control masks, gas masks, breathing masks, procedural masks, nuisance masks and the like.

In one embodiment, said surgical mask 1003 is cut to an appropriate size by the user with scissors or another suitable blade or cutting apparatus. While not required, the user may find it convenient to shape the filter in a manner that it will not “bunch up” in the oral respirator 1000 during use, facilitating a better seal between the air chamber and the environmental air.

In another embodiment (not depicted), said filter 1003 comprises other filtering media suitable for removing a sufficient amount of contaminants from the air to render it safely breathable to the user. In another embodiment, filter 1004 comprises activated carbon, a cotton material, a fibrous cotton material, a fibrous cotton-like material or the like. In yet another embodiment, filter 1003 comprises a material treated with a fungicide, microbicide or other composition that interferes with or reduces the activity, presence (e.g. kills), or reproduction of harmful microbial organisms.

Referring again to the embodiment depicted in FIG. 1, oral respirator 1000 comprises a filter 1003 with an exposed surface area of from about 5 to about 100 square inches, preferably from about 6 to about 24 square inches, and more preferably from about 6 to about 12 square inches of exposed surface area. As used in this specification, exposed surface area shall mean the surface area of the filter 1003 that directly communicates with environmental air.

By way of illustration, one may use tie-on or ear-loop type surgical or nuisance masks. Although not necessary, or even preferable, surgical, surgeon's or procedural masks used with the present invention may be optionally fitted with nosepieces or padding. Optionally, surgical masks may have properties such as latex-free, hypoallergenic, fiberglass-free, anti-fog, anti-reflection, fluid resistant, multiple-ply, odorless, soft, non-irritating and the like.

Preferably, surgical masks 1003 used with the present invention have a bacterial filtration efficiency of greater than about 99% as measured by the modified or standard Greene and Vesley test methods, in vitro testing, or other test method and indicated with industry wide markings such as CE. Preferably, surgical masks 204 used with the present invention have a particulate filtration efficiency of greater than about 95%, more preferably greater than about 97%, and most preferably greater than about 99%, as measured by a particle challenge study using a 0.2 micron latex sphere at a flow rate of 28.3 liters per minute and/or indicated with industry wide markings by CE. Other preferable filtration standards include those endorsed by the Centers for Disease Control (CDC) or equivalent European regulatory agencies (reference is made to EC Directive 89/686). In some aspects, the filtration efficiency protects the wearer from solid and non-solid particles up to twenty times the occupational exposure limit (OEL).

Preferably, the surgical mask 1003 reduces breathing resistance. Preferably, such masks 1003 do not have a semi-rigid self-supporting shape such as molded masks or cone masks. (Reference is made, for example, to 3M Aseptex 1800+NL fluid resistant molded mask or N95 particulate respirator (“SARS Mask”)).

A variety of surgical masks are known within the art and may be suitably employed as a filter in the present invention. By way of demonstration, but not limitation, in some embodiments, one may use a surgical mask according to the teachings of U.S. Pat. No. 4,419,993 (antifogging surgical mask), U.S. Pat. No. 4,355,637 (surgical masks), U.S. Pat. No. 6,281,515 (Lightweight radiation protective garments), U.S. Pat. No. 5,937,445 (One piece surgical mask and cap), U.S. Pat. No. 5,803,075 (Surgical mask with adhesive strip to improve breathing), U.S. Pat. No. 4,312,338 (Surgical mask), U.S. Pat. No. 5,596,985 (surgical mask), U.S. Pat. No. 3,884,227 (disposable surgical mask), U.S. Pat. No. 5,596,985 (radiation-curable, cyonoacrylate-containing compositions), U.S. Pat. No. 5,012,805 (Surgical mask barrier apparatus), U.S. Pat. No. 4,469,097 (medical breathing apparatus), U.S. Pat. No. 6,726,795 (radiation-curable, cyonoacrylate-containing compositions), U.S. Pat. No. 6,412,486 (Disposable filtering face mask and method of making same), U.S. Pat. No. 3,834,384 (Surgical mask with adhesive vapor barrier), U.S. Pat. No. 6,216,695 (Disposable surgical face mask with retractable eye shield), U.S. Pat. No. 4,790,307 (Disposable surgical mask having a self-contained supply of anti-bacterial material), U.S. Pat. No. 4,635,628 (Surgical mask with improved moisture barrier), U.S. Pat. No. 5,783,502 (Virus inactivating coatings), U.S. Pat. No. 5,694,927 (Disposable mask and suction catheter), U.S. Pat. No. 6,237,596 (Disposable mask and suction catheter), U.S. Pat. No. 6,152,137 (Pliable and resilient sealing pad), U.S. Pat. No. 5,585,186 (Coating composition having anti-reflective, and anti-fogging properties), U.S. Pat. No. 5,753,373 (Coating composition having anti-reflective, and anti-fogging properties), U.S. Pat. No. 5,873,931 (Coating composition having anti-reflective, and anti-fogging properties), U.S. Pat. No. 5,900,258 (Anti-bacterial coatings), U.S. Pat. No. 6,365,169 (Polymeric broad spectrum antimicrobial coatings), and U.S. Pat. No. 5,997,621 (Coating composition having anti-reflective, and anti-fogging properties).

As well known to those skilled in the industry, there are a myriad of manufacturers and varieties of suitable masks 204. There are many distributors and suppliers of these masks. By way of example, but not limitation, 3M manufactures many suitable masks 1003. Exemplary of suitable models are 3M 1816 standard-tie surgical mask, 3M 1826 standard earloop face mask, 3M 1862 health care respirator, and 3M 1863 health care respirator.

By way of further example, suitable masks 1003 are manufactured by and/or distributed under brand names such as Moinlycke Barrier, Sultan Chemists USA, Crosstex and Kimberly-Clark. By way of further example, Henry Schein surgeons masks would be well suited for use with the present invention.

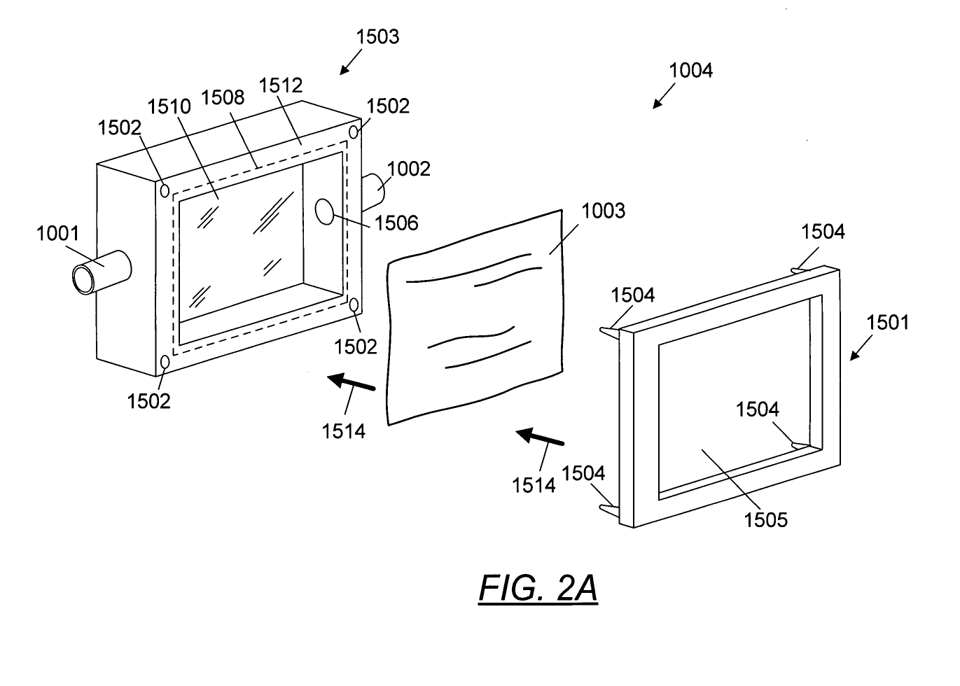

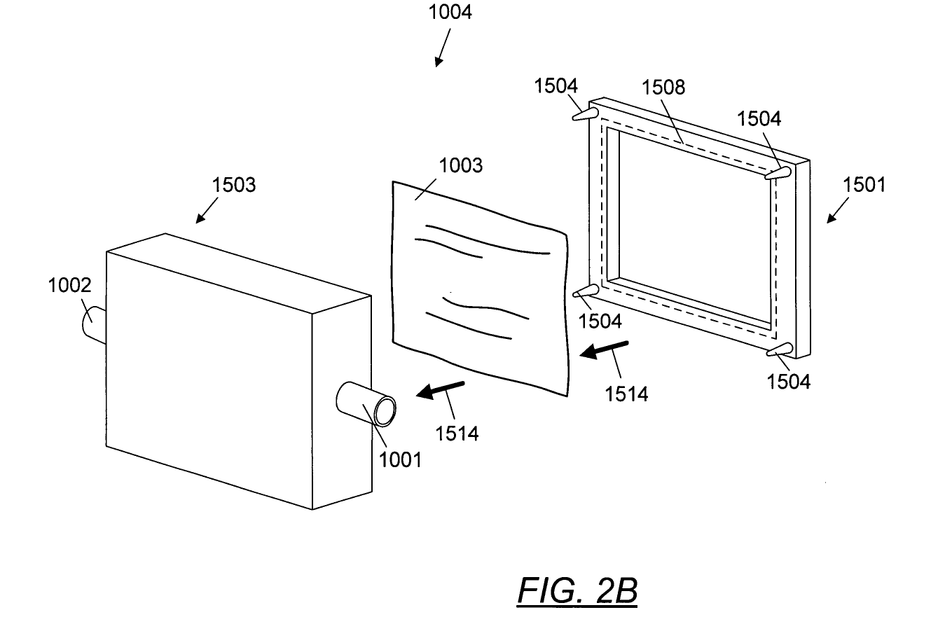

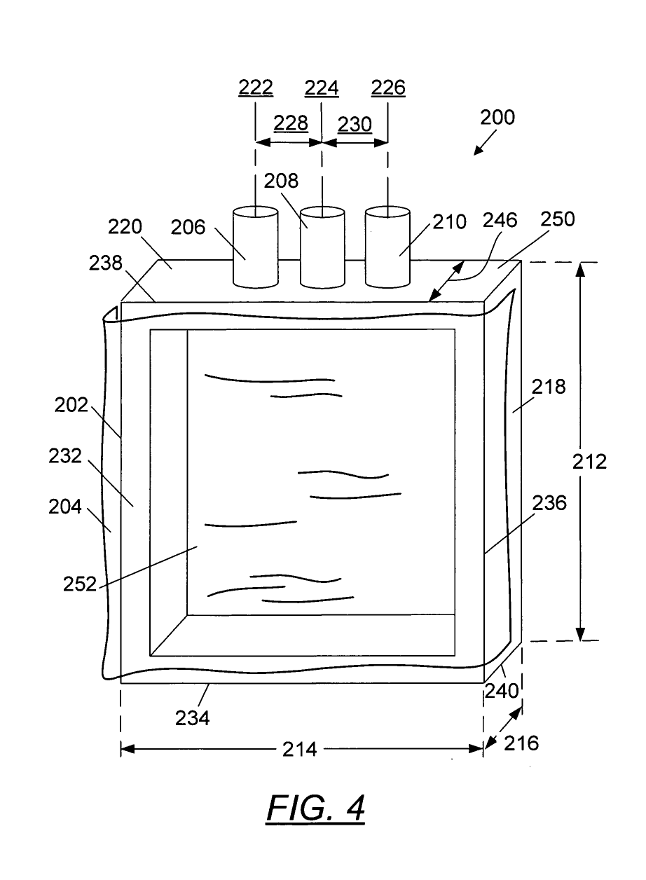

Referring again to the embodiment depicted in FIG. 1, oral respirator device 1000 further comprises a housing 1004 for receiving and confining said filter 1003. Said housing 1004 comprises an air chamber (hidden from view but may be seen in FIG. 2A), a first air inlet 1001, a second air inlet 1002, and a means of securing said filter 1003. In a preferred embodiment, the air chamber comprises a volume of from about 5 to about 1000 cubic inches, preferably from about 5 to about 500 cubic inches, and more preferably from about 5 to about 100 cubic inches. Dimensions of housing 1004, in one embodiment, are as described for the housing depicted and described with reference to FIG. 4 herein.

Referring again to the embodiment depicted in FIG. 1, oral respirator 1000 further comprises oral interface 1006 which enables a user to breathe through said oral respirator 1000 breathable air with particulates having been filtered therefrom. In the embodiment depicted, said oral interface 1006 comprises a tubular member 1009 operatively connected to said housing 1004 at first air inlet 1001 and second air inlet 1002. In one embodiment, said tubular member 1009 comprises a first opening 1031, a second opening 1032, and a third opening 1033, a first one-way valve 1024 operatively connected to said tubular member 1009, and a second one-way valve 1026 operatively connected to said tubular member 1009. Third opening 1033 comprises a mouthpiece 1005 in some embodiments. Any suitable mouthpiece 1005 known to one skilled in the art may be used. As will be apparent, said mouthpiece 1005 preferably has at least four openings, two for connection to the tubular member, one for exhalation and one for inhalation through the oral cavity (mouth). In a preferred embodiment of the mouthpiece 1005, at least one of such openings comprises, or is operably connected to, a one-way air valve. In some embodiments of the mouthpiece 1005, two or more openings comprise, or are operably connected to, one-way air valves. In other embodiments, valves 1026 and 1024 comprise bidirectional air valves. In yet other embodiments, valves 1026 and 1024 comprise respiratory air valves.

In one embodiment of oral respirator 1000 depicted in FIG. 1, said first one-way valve 1024 is disposed along said tubular member 1009 and communicating with said third opening 1033 (e.g. mouthpiece 1005). Similarly, said second one-way valve 1026 is disposed along said tubular member 1009 and communicating with said third opening 1033 (e.g. mouthpiece 1005). In another embodiment (not depicted), said first one-way valve 1024 is disposed at first opening 1031 of tubular member 1009 and communicating with said first air inlet 1001. Similarly, said second one-way valve 1026 is disposed at second opening 1032 of tubular member 1009 and communicating with said second air inlet 1002.

In one embodiment, approximately one-half the distance between the first end 1001 and the second end 1002 of tubular member 1009 is disposed a mouthpiece 1005 comprising 3 one-way air valves 1022, 1024, 1026. In one embodiment, at least one of the one-way air valves 1022, 1024, 1026 is a diaphragmatic valve. In another embodiment, all three one-way air valves 1022, 1024, 1026 are diaphragmatic air valves.

In another embodiment (not shown), a connection piece is operably attached between the first end of the oral interface and the housing. In one embodiment, said connection piece further comprises a valve to control directional airflow.

In yet another embodiment of oral respirator 1000 (not depicted), said housing 1004 comprises a first air inlet and said oral interface 1006 comprises a tubular member with a first opening, a second opening, and a first one-way valve operatively connected to said first opening of said tubular member and said first air inlet of said housing 1004. Said second opening of said tubular member is inserted into the oral cavity of the user to enable inhalation of breathable filtered air from the air chamber. With this configuration, the user simply exhales through the mouth without using the oral interface 1006.

In one embodiment of oral respirator 1000 depicted in FIG. 1, said third opening 1033 of said oral interface 1006 comprises mouthpiece 1005 to provide a comfortable interface for the user. In one embodiment, mouthpiece 1005 comprises a fourth opening wherein the third opening is inserted into the user's mouth and is in fluid flow communication with said fourth opening which is used to facilitate exhalation. In one embodiment, a third one-way valve is operably connected o said fourth opening. In the embodiment depicted, a third one-way valve 1022 is disposed on mouthpiece 1005 at location 1020. During exhalation, air flows outwardly from one way air valve 1022 in direction 1028. During inhalation, breathable air flows from the chamber of the housing into mouthpiece 1005 via tubular member 1009 in the direction of 1030. In embodiments with one-way air valves, breathable air flows from the chamber of the housing into mouthpiece 1005 via tubular member 1009 and through one-way air valves 1024, 1026 in the direction of 1030.

As will be apparent, one way air valves 1022, 1024 and 1026 function to restrict airflow and provide a uni-direction path of airflow. In some embodiments, one way air valves 1022, 1024 and 1026 comprise diaphragmatic valves. Thus, one way air valves 1024 and 1026 permit airflow of breathable filtered air in the direction of 1030 for inhalation of by the user, while preventing exhaled air from flowing into the air chamber of the housing. Similarly, one way air valve 1022 permits airflow of exhaled air in the direction of 1028 by the user, while preventing unfiltered air from being inhaled by the user via mouthpiece 1005.

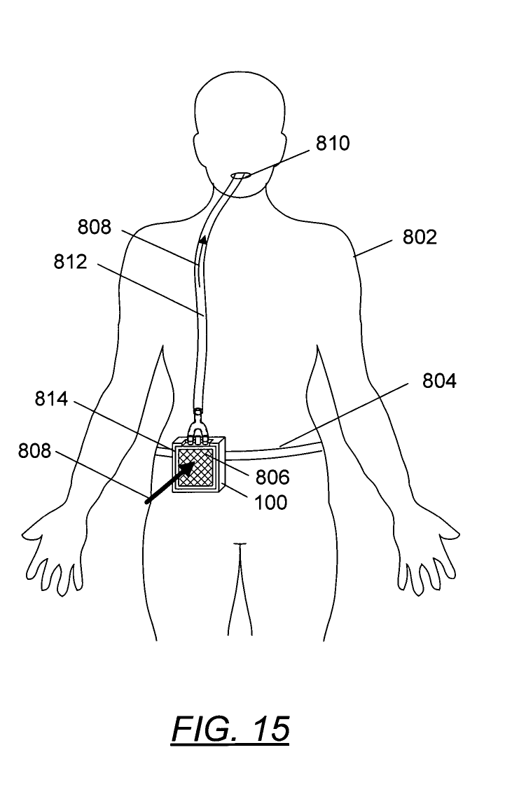

In another embodiment of oral respirator 1000 (not shown), a plurality of flush or recessed air inlet apertures are disposed about the housing similar to the embodiment depicted in FIG. 15. With a plurality of air inlet apertures that may be used, the user may operably connect the oral interface in a manner that maximizes comfort and ease of use. The air inlet apertures that are not operably connected to the oral interface are sealed or capped to prevent the inflow of environmental air into the air chamber.

FIGS. 2A and 2B depict housing 1004 of FIG. 1 from different angles. Referring to FIGS. 2A and 2B, housing 1004 comprises a mated counterpart enclosure comprising a male portion 1501 and a female portion 1503. As will be apparent, and as depicted in FIG. 1, while in use, the male portion 1501 is disposed within, e.g. “mated,” with said female portion 1501 such that an air chamber 1510 is formed therebetween. A filter 1003 is removably secured between the male portion 1501 and female portion 1503 about an opening 1505 in the male portion 1501 such that it is disposed between the intake for environmental air and the air chamber 1510 with the filtered breathable air. Such configuration will hereinafter be referred to as the housing's “mated configuration.”

Referring again to FIGS. 2A and 2B, filter 1003 is removably secured between the male portion 1501 and female portion 1503 via prongs 1504 inserted in the direction 1514 into apertures 1502 and forming a compression or pressure seal between the male portion 1501 and female portion 1503 of the housing 1004. Prongs 1504 comprise elongated, angled or tubular protrusions of from about one to about 10 millimeters in length. Apertures 1502 comprise recesses or holes that correspond to said prongs in location and size in a manner that a press-fit or snap-fit may be obtained by the user. As will apparent, an inverse relationship may be used with apertures 1502 disposed about portion 1501 and prongs 1504 disposed about portion 1503.

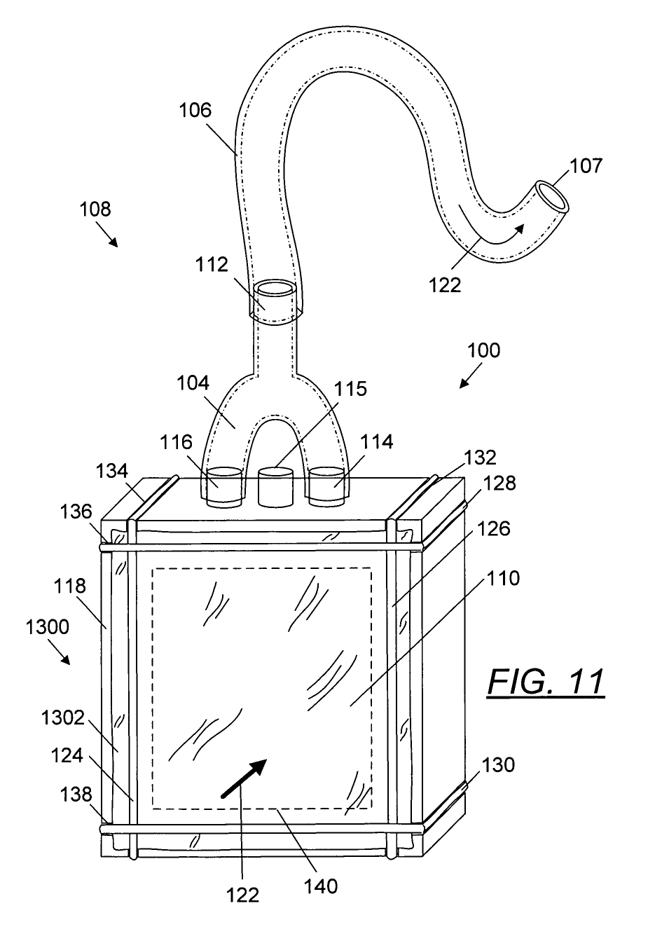

Alternatively, in another embodiment (not depicted), a compression seal may be formed with a rib and groove configuration disposed along broken line 1508 of FIG. 2A. Such means of sealing with prong-hole or rib and groove configurations are well known in the art. Alternatively, any means known to one skilled in the art may be used to removably secure the housing in its mated configuration and secure filter 1003 in place. For example, a crude method employing simple elastic bands or ties is depicted and described with reference to FIG. 11.

Referring to FIGS. 2A and 2B, in using oral respirator 1000, the user disposes filter 1003 on first side 1512 of female portion 1503 of the housing in direction 1514 in such a manner that the filter 1003 lies flat across the surface of first side 1512 and does not obstruct apertures 1502. Male portion 1501 is thereafter disposed on top of filter 1003 in direction 1514 in such a manner that prongs 1504 are inserted within apertures 1502 and form a compression seal therewith. Thus, filter 1003 directly communicates with the male 1501 and female 1503 portions of the housing 1004 when in use. A portion of filter 1003 is exposed to the environmental air through opening 1505 in the male portion 1501 of the housing.

Environmental air enters the air chamber 1510 via filter 1003 during inhalation thereby being filtered into breathable air. Breathable air exits the air chamber 1510 via air inlets 1001 and 1002 into the oral cavity of the user via oral interface 1006. Air inlet 1002 may be protruding as depicted, but such configuration is not required. Air inlets 1001 and 1002 may be recessed or flush with the surface of the housing wall. It is preferred that air inlet 1002 be in fluid flow communication with the air chamber 1510 via aperture 1506. Similarly, air inlet 1001 is preferably in fluid flow communication with the air chamber 1510.

Referring again to FIGS. 2A and 2B, filter 1003 is secured in place—means plus function language

Referring again to FIGS. 2A and 2B, one may use a surgical mask as a filter 1003. The filter 110 may be, but is not required to be, cut by the user (with scissors for example) to an appropriate size that may be accommodated by the housing 1004. Use of a common surgical mask as a filter 1003 is one of the advantageous features of the present invention, eliminating the necessity of specialized filters and allowing the user to incorporate items commonly available in a hospital, industrial location, emergency vehicle, clinic or medical office.

Referring again to FIG. 1 and the embodiment depicted, oral interface 1006 comprises a tubular member 1009 operatively connected to said housing 1004, wherein said tubular member 1009 comprises a first opening 1031, a second opening 1032, and a third opening 1033. Said third opening comprises mouthpiece 1005 and is disposed between said first opening 1031 and said second opening 1032, being operably connected thereto via a first one-way valve 1024 and a second one-way valve 1026, wherein said mouthpiece 1005 enables a user to breathe through said respirator 1000.

In one embodiment depicted in FIG. 1, oral interface 1006 comprises a tubular member 1009 such as a plastic tube, e.g. a surgical tube, with a lumen diameter of from about one to about twenty millimeters, preferably from about 5 to about 10 millimeters. In one embodiment, the tube permits one way airflow through the tube upon inhalation by the user (reference is made to FIG. 11A). In another embodiment, said tube is bifurcated and permits bidirectional airflow (reference is made to FIG. 11B). One portion of such bifurcated tube may be used to transport filtered breathable air upon inhalation by the user while the second portion may be used to transport exhaust air when the user exhales. In one embodiment as depicted, there is a mouthpiece 1005 affixed to the plastic tube.

In another embodiment (not shown), a protective shield is removably disposed about the second end of the oral interface which protective shield may be used to cover and protect the second end (or mouthpiece) of the oral interface from debris and germs when not in use.

In another embodiment (not shown), an attachment mechanism is operatively connected to the oral interface such that it may be removably affixed to the user's clothing. As will be apparent, one may use any appropriate attachment mechanism known to those skilled in the art. By way of illustration, but not limitation, one may use a pin, magnet, clasp, clip, tape, portion of Velcro or the like. Preferably, the attachment mechanism will not damage the user's clothing and may be easily replaced if it becomes inoperable. As will be apparent, this may be used in combination with, or as an alternative to, the protective shield previously described as a way to protect the second end/mouthpiece of the oral interface from debris and germs when not in use.

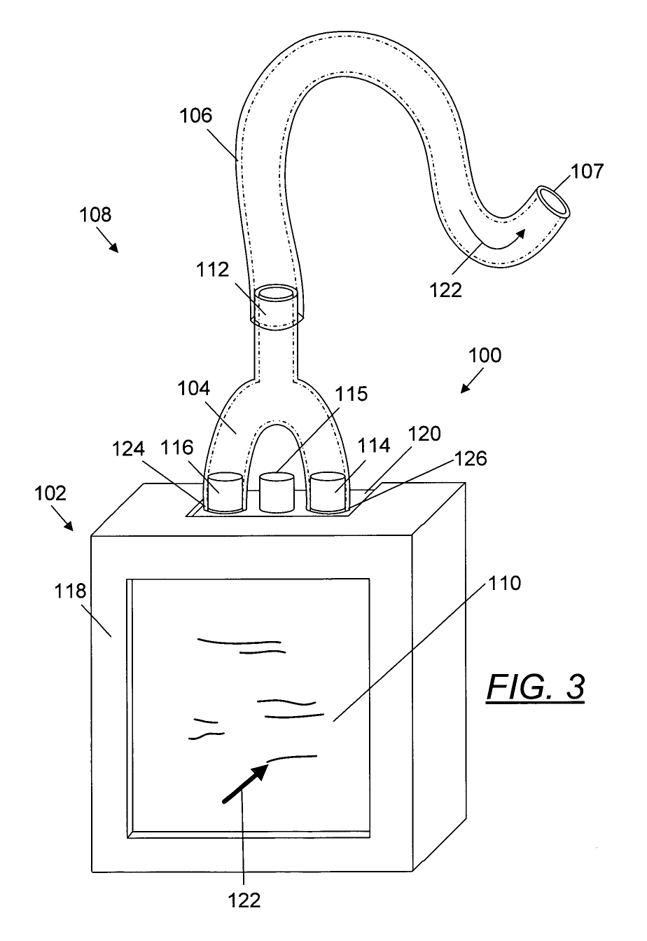

FIG. 3 depicts another embodiment of an oral respirator device 100 for mask-free filtering of breathed air. In its broadest context, referring to FIG. 3 and the embodiment depicted therein, the present invention consists of a housing 102, an oral interface 108 and a filter 110 arranged directly upstream in a direction of airflow 122.

Referring again to the embodiment depicted in FIG. 3, oral respirator device 100 comprises a filter 110 removably disposed within said oral respirator 100, wherein said filter 110 is a surgical mask, a housing 102 for receiving and confining said filter 110 comprising an air chamber, a first air inlet, a second air inlet, and a means of securing said filter, an oral interface 108 comprising a tubular member operatively connected to said housing 102, wherein said tubular member comprises a first opening, a second opening, and a third opening, a first one-way valve operatively connected to said first opening of said tubular member, a second one-way valve operatively connected to said second opening of said tubular member, wherein said oral interface 108 enables a user to breathe through said respirator 100, and wherein said oral respirator 100 filters particulates from breathed air.

Referring again to FIG. 3, the housing 102 comprises a mated counterpart enclosure comprising a male portion 120 and a female portion 118. As will be apparent, and as depicted in FIG. 3, while in use, the male portion 120 is disposed within, e.g. “mated,” with said female portion 118 such that an air chamber (hidden from view) is formed therebetween. A filter 110 is removably secured about the opening in the female portion 118, e.g. a faceplate, such that it is disposed between the intake for environmental air and the air chamber with the filtered breathable air. Such configuration will hereinafter be referred to as the housing's “mated configuration.”

Referring again to FIG. 3, oral interface 108 comprises a tubular member 106 operatively connected to said housing 102, wherein said tubular member 106 comprises a first opening 107, a second opening 124, and a third opening 126. In one embodiment, said tubular member further comprises a first one-way valve operatively connected to said second opening 124 of said tubular member and a second one-way valve operatively connected to said third opening 126 of said tubular member. Said oral interface 108 enables a user to breathe through said respirator 100.

Referring again to FIG. 3 and the embodiment depicted, oral interface 108 comprises a tubular member. In one embodiment, said tubular member comprises a plastic tube (“tube”), e.g. a surgical tube, with a lumen diameter of from about one to about twenty millimeters, preferably from about 5 to about 10 millimeters. In one embodiment, said tube permits one way airflow upon inhalation by the user. In another embodiment, said tube is bifurcated and permits bi-directional airflow. One portion of such bifurcated tube may be used to transport filtered breathable air upon inhalation by the user while the second portion may be used to transport exhaust air when the user exhales. In one embodiment (not depicted), there is a mouthpiece affixed to the plastic tube.

In another embodiment, not shown, the housing 102 is a unitary piece comprising one part rather than male and female portions of the housing.

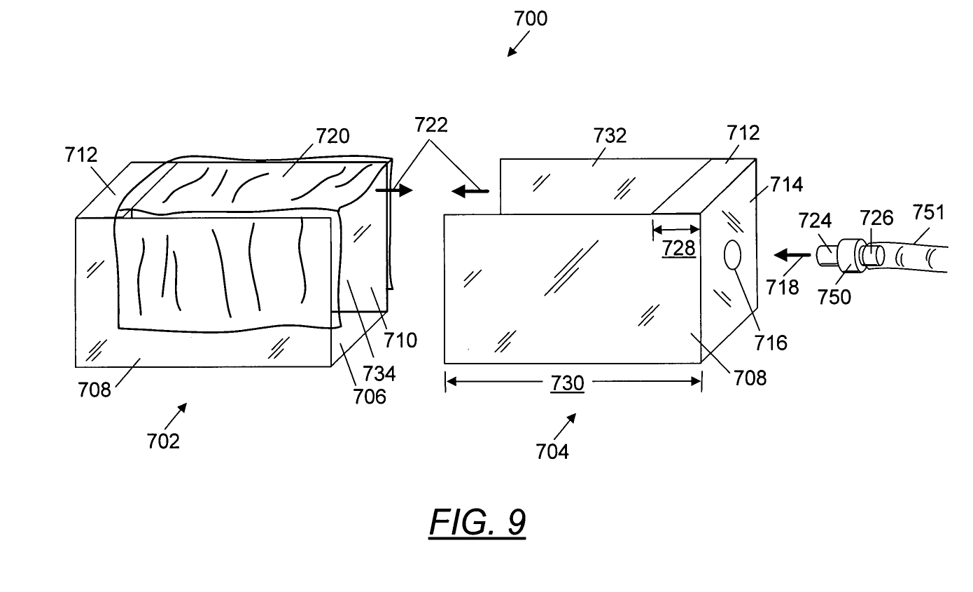

In another embodiment, such as ones depicted in FIGS. 9 and 15, the male and female portions of the housing are identical.

Referring again to FIG. 3, the oral interface 108 comprises a male portion 104 and a female portion 106 operatively connected at 112 with a substantially airtight seal. In the embodiment depicted, the male portion 104 is bifurcated to facilitate an operative connection with the housing at air inlets 114 and 116. The female portion 106 is inserted into the oral cavity, e.g. mouth, of the user at end 107.

In another embodiment, depicted in FIGS. 11A and 11B, the oral interface is a unitary piece.

Optionally, air inlet 115 may be capped when not in use to prevent unfiltered air from entering the air chamber and mixing with the breathable filtered air. It is to be understood that various combinations of connecting air inlets 114/115/116 with the oral interface 108 are possible. It is preferred in one embodiment that at least one air inlet must be operatively connected to the oral interface 108. Optionally, and preferably, two (or even three) air inlets may be connected.

FIG. 4 depicts an embodiment of the housing with the male portion 200 of the housing visible. In one embodiment, said housing 200 in its mated configuration essentially comprises a rectangular plastic box with a length 212 of from about at least about three inches to at least about eighteen inches and a width 214 of at least from about 3 inches to about 12 inches. In a preferred embodiment, said housing in its mated configuration comprises a rectangular box with a length 212 of about 5 inches and a width 214 of about 4 inches. The depth 216 of said housing in its mated configuration is from about one-half inch to about 8 inches, preferably from about one-half inch to about 2 inches, more preferably from about one-half inch to about one and one-half inches. The interior of said rectangular box comprises the air chamber.

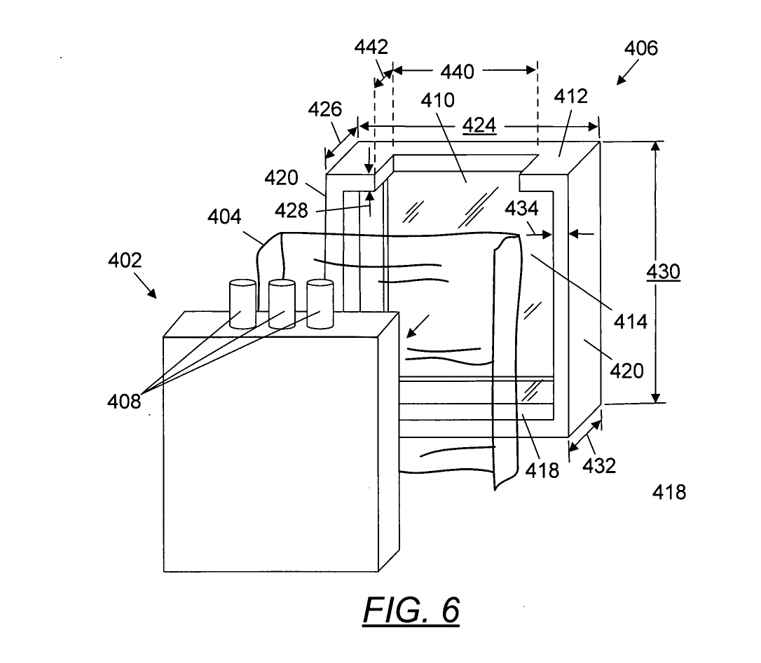

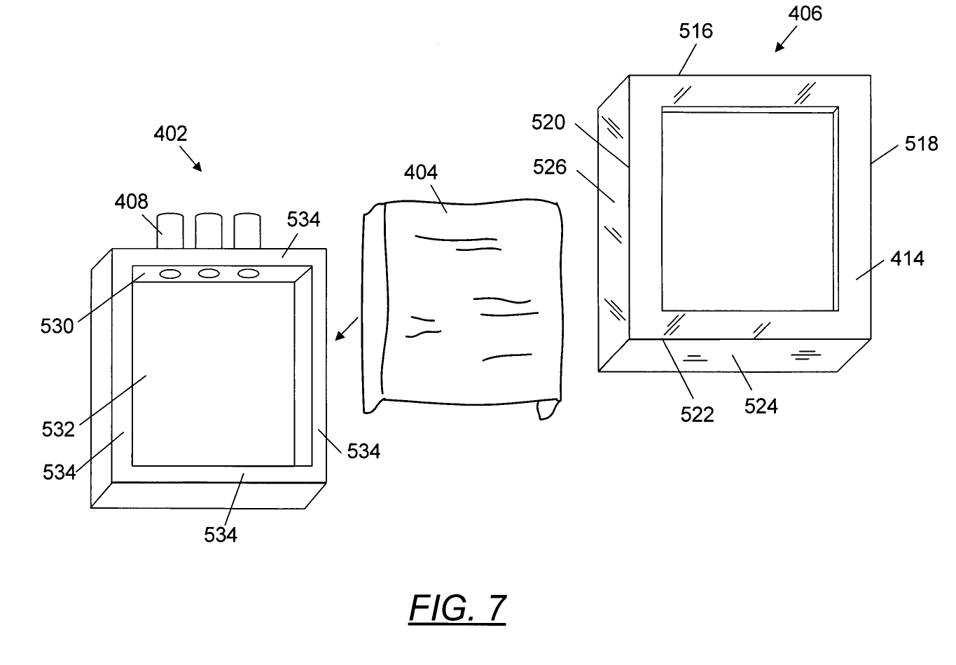

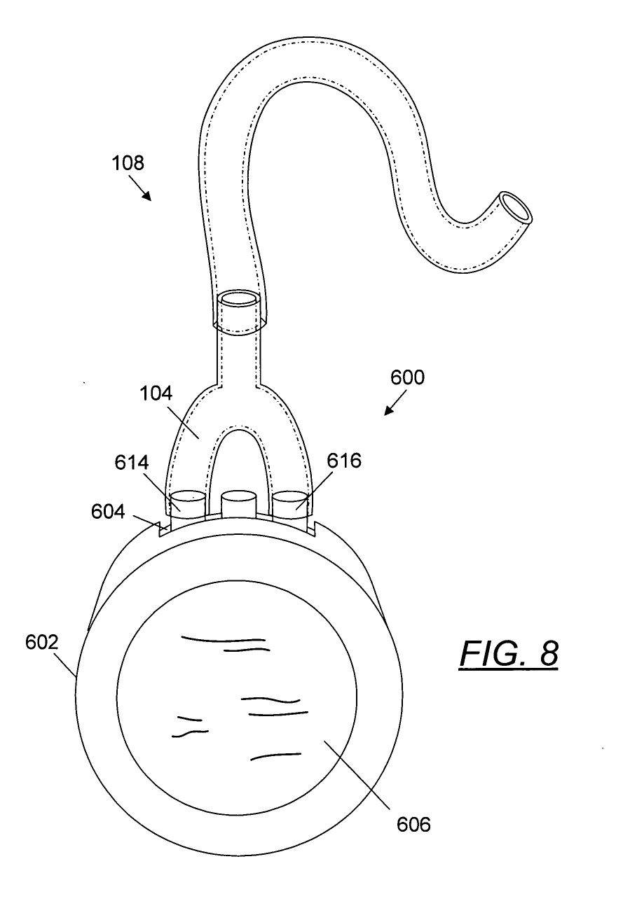

FIGS. 6 and 7 depict both portions of the housing depicted in FIG. 3 and how such parts are mated together. FIG. 8 depicts an alternate embodiment of the device in FIG. 3. In some embodiments, said housing is rectangular shaped as depicted in FIGS. 3, 6 and 7. In other embodiments, the housing is round as depicted in FIG. 8. In yet other embodiments (not shown) the housing is oblong shaped. In yet other embodiments (not shown), a non-geometric shape is used.

It is should be understood that the shapes and dimensions may vary in infinite combinations, however, it is preferred that the dimensions be adequate to create an air chamber with a volume adequate to permit comfortable breathing by the user. The air chamber comprises a volume of from about 5 to about 1000 cubic inches, preferably from about 5 to about 500 cubic inches, and more preferably from about 5 to about 100 cubic inches.

Referring again to FIGS. 1 and 3, said housing 1000, 200 is comprised of a high density plastic. In another embodiment, a polycarbonate material may be utilized. In some embodiments, one may utilize a lightweight metal or metal alloy. In other embodiments, a softer polymeric material may be used.

Referring again to FIG. 4, the peripheral edge of the male portion 200 of the housing comprises a top edge 238, a bottom edge 234 and opposed edges 202, 236. Integrally connected to each peripheral edge is a vertically-extending side wall, to wit, a top side wall 220, a bottom side wall 240 and two opposing side walls 218 (other hidden from view). Preferably, the side walls 218/240/220 of the male portion of the housing 200 are formed via a process, for example injection molding, that produces the male portion as a single component, however, the side walls may be operatively connected by any appropriate means of adhesion known to those skilled in the art. These side walls preferably form an angle 242, 244 with the surface of peripheral edge of the male portion of from about 80 to about 110 degrees, more preferably about 90 degrees.

Referring again to FIG. 4, the top and bottom side walls of the male portion 200 of the housing preferably have a length 214 of from about 3 inches to about 12 inches and, more preferably, from about 4 inches to about 6 inches. The top 220 and bottom 240 side walls of the male portion of the housing preferably have a width 246 of from about one-half inch to about 8 inches and, more preferably, from about one-half inch to about 2 inches. The top 220 and bottom 240 side walls of the male portion of the housing preferably have a thickness of from about 0.25 millimeters to about 4 millimeters and, more preferably, from about one to about two millimeters.

Referring again to FIG. 4, the opposing side walls 218 of the male portion 200 of the housing preferably have a length 212 of from about 3 inches to about 18 inches and, more preferably, from about 4 inches to about 8 inches. The opposing side walls 218 of the male portion 200 of the housing preferably have a width 216 of from about one half inch to about 8 inches and, more preferably, from about one-half to about 2 inches. The opposing side walls 218 of the male portion of the housing preferably thickness of from about 0.25 millimeters to about 4 millimeters and, more preferably, from about one to about two millimeters.

As will be apparent, the surface of the peripheral edge 202/238/234/236 of the male portion 200 of the housing is adapted to engage the filter 204 and to prevent it from moving longitudinally or transversely.

Referring again to FIG. 4, said top side wall 220 of the male portion 200 of the housing has an outer surface 250 and an inner surface (hidden from view). (Reference is also made to FIG. 7 where the same may be viewed from a different perspective.) In one embodiment (not shown), disposed along the outer surface of the top side wall 220 of the male portion 200 of the housing, and integrally connected thereto, is at least one aperture 206/208/210 that may function as an air inlet permitting unidirectional or bidirectional air flow to and/or from the air chamber. In one embodiment, said air inlet is disposed in the center of said top side wall. It is preferred that aperture 206/208/210 be in fluid flow communication with said air chamber within said housing.

In one embodiment depicted in FIG. 4, at least three such apertures 206/208/210 are disposed along from the outer surface 250 of the top side wall 220 of the male portion 200 of the housing. (Reference is also made to FIG. 7 where the same may be viewed from a different perspective.) In some embodiments, said aperture 206/208/210 is configured to operatively connect the oral interface (108 of FIG. 3) with a substantially airtight seal. In some embodiments, said apertures 206/208/210 comprise a hole in the top side wall 220. In some embodiments, said apertures 206/208/210 comprise a tubular protrusion from the top side wall 220. In some embodiments, said apertures 206/208/210 comprise a threaded hole in the top side wall 220 such that an adapter, fitting or oral interface may be screwed into the apertures 206/208/210. In yet other embodiments, said apertures 206/208/210 comprise depressions or other openings that provide the desired function of permitting airflow from the oral interface into the air chamber of the housing. In yet other embodiments, said apertures 206/208/210 comprise valves.

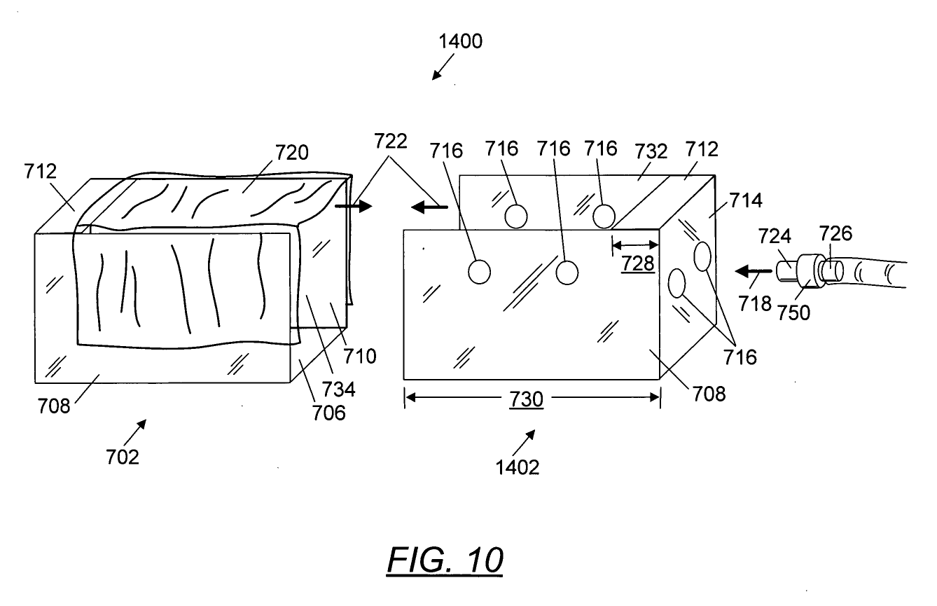

As will be apparent to those skilled in the art, in some embodiments, a suitable connector may be used to operatively connect the air inlet aperture and the oral interface. By way of illustration, but not limitation, said connector may be one as depicted at 104 of FIG. 3. By way of further illustration, in cases where the oral interface is configured as shown in FIGS. 11A, 11B and 10 as a unitary piece, a connector (not shown) in the way of a fitting or adapter, may be used to operatively connect the housing 200 and the oral interface (108 of FIG. 3) with a substantially airtight seal at 114 and 116. By way of example, a fitting such as 750 of FIG. 9 may be used.

In one embodiment depicted in FIG. 4, said air inlets 206/208/210 are disposed along the outer surface 250 of the top side wall 220 of the male portion 200 of the housing at a distance 228, 230 between their respective centerpoints 222/224/226 of from about 2 millimeters to about seventeen inches, preferably from about one-quarter inch to about one and one-half inches, and more preferably from about one-half to about one inch.

Referring again to FIG. 4, said top side wall 220 of the male portion 200 of the housing has an outer surface 250. Disposed along and the outer surface 250 of the top side wall 220 of the male portion 200 of the housing, and integrally connected thereto, is at least one peripherally disposed and outwardly facing protrusion 206/208/210 that may function as an air inlet permitting unidirectional or bidirectional air flow and is configured for selectively snap-engaging the oral interface. As will be apparent, snap-fitting comprises only one of a myriad of operative connection methods. Press or screw fitting would also be suitable in this application. In a preferred embodiment depicted in FIG. 4, at least three such peripherally disposed protrusions 206/208/210 are disposed along the outer surface 250 of the top side wall 220 of the male portion 200 of the housing.

In embodiments where three air inlets are employed, they may be used in any combination to facilitate inhalation and exhalation. In one embodiment, two air inlets are used for inhalation and one for exhalation. In another embodiment, one air inlet is used for inhalation, one for exhalation, and one is capped as a “spare” should one the air inlets become inoperable. As will be apparent, there are many combinations that one may devise in building and using the present invention, all of which are to be considered within the scope of the present invention.

In other embodiments, not shown, said apertures 206/208/210 may be recessed.

Referring again to FIG. 4, surface 232 of the male portion 200 of the housing comprises a solid frame surrounding an opening 252, forming an open face plate 232, as depicted in FIG. 4. This opening 252 exposes the filter 204 disposed between the open face plate 232 and the air chamber (not visible, however, reference is made to 248).

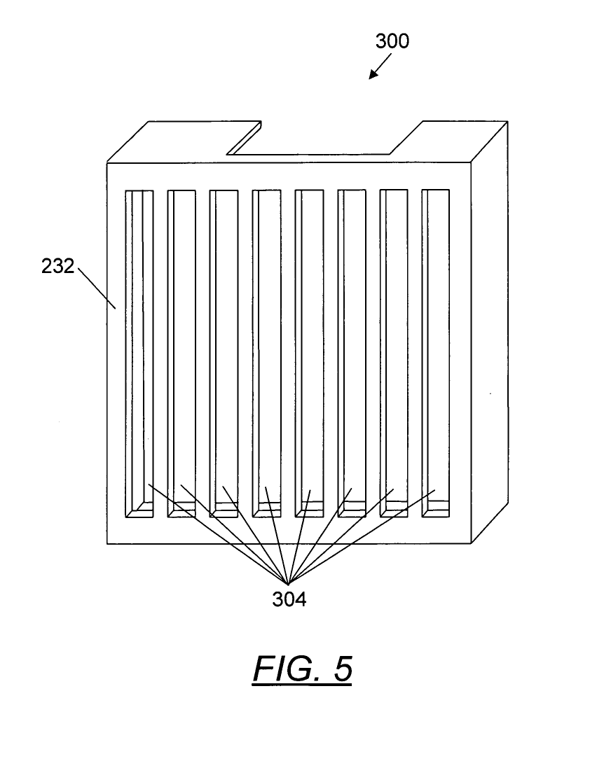

FIG. 5 depicts another embodiment of a portion of the housing 300. In another embodiment 300 depicted in FIG. 5, said surface 232, e.g. face plate, comprises a plurality of open vents 304 with an exposed surface of from at least from about five to at least about ninety percent, preferably at least about fifty per cent and more preferably at least about seventy per cent as depicted in FIG. 5. This plurality of open vents 304 expose a portion of the filter (not shown) allowing it to communicate with the environmental air. The filter is disposed between the face plate 232 (communicating with its underside) and the air chamber (not visible).

FIGS. 6 and 7 depict both portions of the housing depicted in FIG. 3 and how such parts are mated together. The female portion 406 of the housing is substantially the same material, shape and dimension as the male portion 402 of the housing, being slightly smaller such that it communicates and interlocks, e.g. “mates,” with the male portion and forms a substantially airtight seal therebetween.

Referring again to FIG. 6, in a preferred embodiment, the filter 406 is removably secured about the opening in the faceplate such that it is disposed between the intake for environmental air and the air chamber with the filtered breathable air. As will be apparent, other suitable arrangements may be used to accomplish the desired filtering effect.

In one embodiment depicted in FIG. 6, the female portion 406 of the housing differs from the male portion 402 in at least one way. In lieu of the air inlet apertures, e.g. protrusions 408, the female portion 406 comprises an orifice 410, e.g. a cutout, disposed along the top side wall 412 of the female portion 406 of the housing and is configured such that it partially or completely surrounds the air inlets or protrusions 408 and facilitates the mating of the male 402 and female 406 portions of the housing. In a preferred embodiment depicted in FIG. 6, the cutout 410 is substantially “U-shaped.”

In other embodiments (not shown), the face plate of the female portion of the housing comprises openings substantially similar as those described for the male portion of the housing (reference is made to 304 of FIG. 5 and 252 of FIG. 4).

In some embodiments (not depicted), two filters are used and the housing comprises two openings similar to 304 of FIG. 5 or 252 of FIG. 4 to expose the filters to the environmental air and provide greater inflow of air into the air chamber. It is not required that both openings be identical. For example, one opening may be similar to 304 of FIG. 5 while the other is similar to 252 of FIG. 4. As another example, the openings may be different sizes or shapes.

In other embodiments (not shown), there is an inverse relationship in the mating of the parts such that the air inlets or protrusions are disposed along the top side wall of the female portion of the housing and the cutout is disposed along the top side wall of the male portion of the housing.

In other embodiments (not shown), the air inlets or protrusions and their respective cutouts are disposed along the bottom or opposing side walls of the male and female portions of the housing respectively.

In other embodiments (not shown), the side walls of the male portion of the housing have a width of from about 1 millimeter to about one inch and when mated with the female portion of the housing, thus causing no overlap or interference with the air inlets or protrusions. As will be apparent, such configuration requires no cutout in the side walls of the female portion. In yet another embodiment, the inverse relationship may be used such that the side walls of the female portion have a width of from about 1 millimeter to about one inch and when mated with the male portion of the housing, causes no overlap or interference with the air inlets or protrusions.

Referring again to FIG. 7, the peripheral edge of the female portion 406 of the housing comprises a top edge 516, a bottom edge 522 and opposed edges 518, 520. Integrally connected to each peripheral edge of the female portion of the housing is a vertically-extending side wall (from face plate 414), to wit, a top side wall (not visible in this view), a bottom side wall 524 and two opposing side walls 526 (other not visible in this view). Preferably, the side walls 524, 526 of the female portion 406 of the housing are formed via an injection molding process that produces the female portion as a single component, however, the side walls may be operatively connected by any appropriate means of adhesion known to those skilled in the art. These side walls preferably form an angle with the surface of peripheral edge of the female portion of the housing of from about 80 to about 110 degrees, more preferably about 90 degrees.

Referring again to FIG. 6, the top 412, bottom side walls 418, and opposing side walls 420 of the female portion 406 of the housing are sized to correspond to and snugly fit together with the male portion 402 of the housing in its mated configuration. As such, the dimensions of such walls 412, 418 and 420 are substantially the same as and varying less than 10 per cent from the dimensions of, the corresponding walls 218/240/220 in the male portion of the housing.

Referring again to FIG. 7, filter 404 is disposed over and communicating with the upper edge 534, creating air chamber 532 which is in fluid flow communication with air inlets 418. Over top is disposed female portion 406 such that the underside of the faceplate 414 communicates with the filter and secures it in place. Reference is also made to FIG. 6.

Referring again to FIG. 6, the cutout 410 has dimensions that allow it to surround the air apertures (reference is made to 206/208/210 of FIG. 4) without encroachment. The shape may comprise any shape that is convenient for the manufacturer or user. In one embodiment, the cutout 410 has a substantially U-shape with rectilinear corners. In other embodiments, the U-shape may take a more rounded appearance. In one embodiment, the length 440 of the cutout 410 is from about 1.4 centimeters to about 5.5 centimeters. In one embodiment, the width 442 of the cutout 410 is from about 0.8 centimeters to about 4 centimeters.

Referring again to FIG. 7, interior of the male portion 402 of the housing is shown, wherein the air chamber 532 is formed beneath filter 404.

In using the present invention, FIGS. 6 and 7 demonstrate how the male and female portions are configured with respect to one another and the filter 404. As will be apparent, the filter 404 is disposed between the male portion 402 and female portion 406 of the housing. The male portion 402 is disposed within the female portion 406 of the housing to form a substantially rectangular box.

FIG. 8 depicts an alternative embodiment of the device 600. As will be apparent, the housing in this embodiment is circular rather than rectangular. Similar to the device 100 depicted in FIG. 3, the female portion 602 of the housing is mated with the male portion 604 of the housing with the filter 606 secured therebetween. The oral interface 108 is operably connected to the air inlets 614, 616.

FIG. 11 depicts an alternative embodiment 1300 of the housing of device 100. In one embodiment depicted, the shape of the housing is a rectangular cube with open side 1302 (there is no wall). As will be apparent, the device is substantially the same as described previously except that the housing 1300 is a unitary piece and the filter 110 is secured to the housing 1300 by straps, ties or rubber bands 124, 128, 126, 130 to the open side 1302 of the housing. The straps, ties or rubber bands fit within small channels 132, 136, 134, 138 that surround the entire periphery of the device. These channels keep the straps, ties or rubber bands from dislocating, thus keeping the filter 110 tightly secured to the housing. In one embodiment, these channels have a diameter of from about one to about 4 millimeters and are disposed at a distance of from about 4 to about 30 millimeters from the outside edge of each side.

Referring again to FIG. 11, as will be apparent, many configurations of the channels 132, 136, 134, 138 are possible to accomplish the securing of the filter 110 to the housing and all are considered to be within the scope of this invention. For example, less or more than four channels may be used, or the channels may be centrally located along each side rather than disposed near the periphery, and the like. Preferably, there are at least two channels disposed such that the intersection of the straps, ties or rubber bands are disposed at an angle of from about 50 to about 120 degrees from one another and keep the filter from moving transversely, longitudinally or vertically.

In one embodiment of device 1300 (not shown), there is a face plate substantially the same as described previously for face plate 232 of FIG. 4. Dashed line 140 depicts where the interior periphery of a faceplate (not shown) may be disposed in such embodiment. Channels and elastic bands may be used to secure the face plate and filter in place. In this configuration, the filter 110 is disposed between the face plate and top of the chamber. In one embodiment, the face plate has channels for secure straps, ties or rubber bands as described with reference to FIG. 11. In one embodiment, the face plate is secured to the housing by a snap-lock mechanism. In another embodiment, the face plate is secured to the housing by a screw mechanism. In one embodiment, the face plate has a picture-frame like shape and has ridges along the bottom surface that fit into the corresponding channels one the housing and keep it from moving transversely, longitudinally or vertically.

FIG. 9 depicts another embodiment of the housing. Referring to FIG. 9, the housing comprises a male 702 and a female 704 portion that are substantially identical to one another. Each portion 702, 704 comprises a cube-like structure with 5 sides. The five sides comprise a bottom panel 706, two opposing side panels 708, 710 that are vertically extending from the plane of the bottom panel 706, a back panel 714 that connects at its peripheral ends with side panels 708, 710, bottom panel 706, and top panel 712. The length 728 of top panel 712 extends only a portion (from about 10% to about 50%, preferably from about 10 to about 25%) of the distance of the length 730 of side panels 708 and 710.

As depicted in FIG. 9, both parts are inversely disposed such that the open sides face one another. The two portions 702 and 704 are operably connected by snapping together in the direction of 722. As will be apparent, an opening 732 is created that is similar to the open face plate in device 100 and an air chamber 734 is formed in the interior of the portions when mated together.

Referring again to FIG. 9, filter 720 is disposed such that it entirely covers the opening 732 and is secured between the two portions 702, 704 when mated together. Thus, when in use, air passes through the filter into the chamber 734, and said filtered air, passes through aperture 716 to the oral interface.

Referring again to FIG. 9, substantially in the center of the back panel 714, is disposed at least one aperture 716, e.g. a hole, along its surface, functioning as the air inlet to the oral interface from the chamber. In some embodiments, said apertures 716 are configured to directly and operatively connect the oral interface, fitting, valve or an adapter 750 with a substantially airtight seal. This may be accomplished with a tubular protrusion or other configuration that will be apparent to one skilled in the art. In some embodiments, said apertures 716 comprise a threaded hole such that an adapter, fitting, valve or oral interface 750 may be screwed into the aperture 716. In yet other embodiments, said apertures 716 comprise a valve such as a unidirectional or bidirectional valve. In yet other embodiments, said apertures 726 comprise depressions or other openings that provide the desired function of permitting airflow to the oral interface from the air chamber of the housing.

Referring again to FIG. 9, adapter, fitting or valve 750 has a first end 724 and a second end 726. First end 724 operatively connects to aperture 716 in the direction of 718 to form a substantially airtight seal. Second end 726 operatively connects to the oral interface 751.

FIG. 10 depicts a modification to the embodiment depicted in FIG. 9. As will be apparent, the embodiment 1400 in FIG. 10 is substantially identical to that in FIG. 9 except that there are two apertures 716 along the surface of the two opposing side panels 708, 710 and back panel 714. (These apertures are substantially the same as apertures 716 of FIG. 7.) These apertures 716 may be disposed along the surface of the two opposing side panels 708, 710 and back panel 714 in any location that is convenient for the manufacturer or user. Applicant believes that a somewhat central location provides the optimum utility.

Referring again to FIG. 9, in some embodiments, a one-way respiratory valve 750 is operably connected to the air inlet hole 716. Such respiratory valves are well-known and any suitable one-way valve may be used. By way of example, but not limitation, one may use a valve according to the teachings of U.S. Pat. No. 4,172,467 (Respirator valve for respirators); U.S. Pat. No. 4,333,453 (Respirator valve); U.S. Pat. No. 6,886,561 (Respiratory valve); or U.S. Pat. No. 6,745,769 (Respiration Bag).

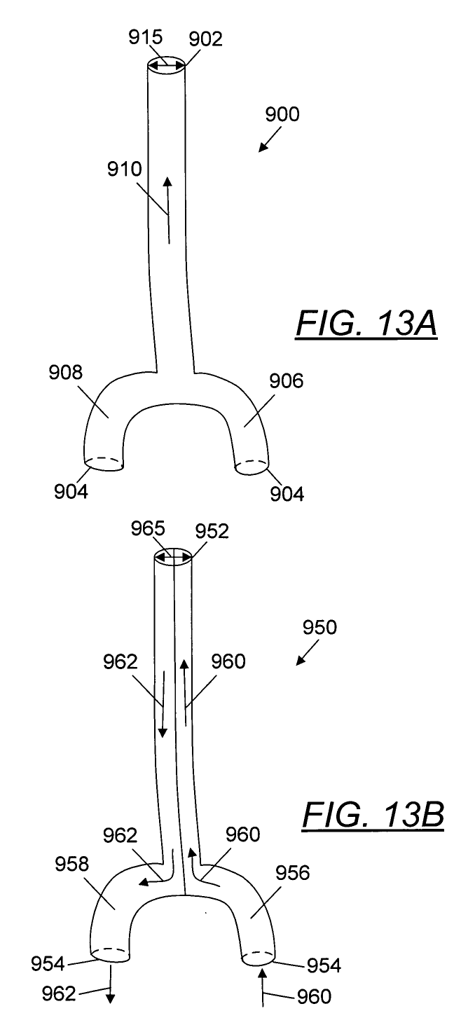

As will be apparent to those skilled in the art, in some embodiments, a suitable connector may be used to operatively connect the air inlet aperture and the oral interface. By way of illustration, but not limitation, said connector may be one as depicted at 104 of FIG. 3 and 750 of FIG. 9. By way of further illustration, in cases where the oral interface is configured as shown in FIGS. 13A and 13B as a unitary piece, a connector (not shown) in the way of a fitting or adapter, may be used to operatively connect the housing 200 and the oral interface (108 of FIG. 3) with a substantially airtight seal.

Referring to FIG. 3, the device further comprises an oral interface 108 for air delivery between the air chamber and the user. FIG. 13A depicts one embodiment of a unidirectional oral interface 900. FIG. 13B depicts one embodiment of a bidirectional oral interface 950. Referring to FIGS. 13A and 13B, the oral interface 900, 950 comprises a first end 904, 954 and a second end 902, 952. The first end 904, 954 of the oral interface 900, 950 is operably connected to the housing (not shown). The second end 902, 952 of the oral interface 900, 950 is inserted into the mouth of the user while the device is in use.

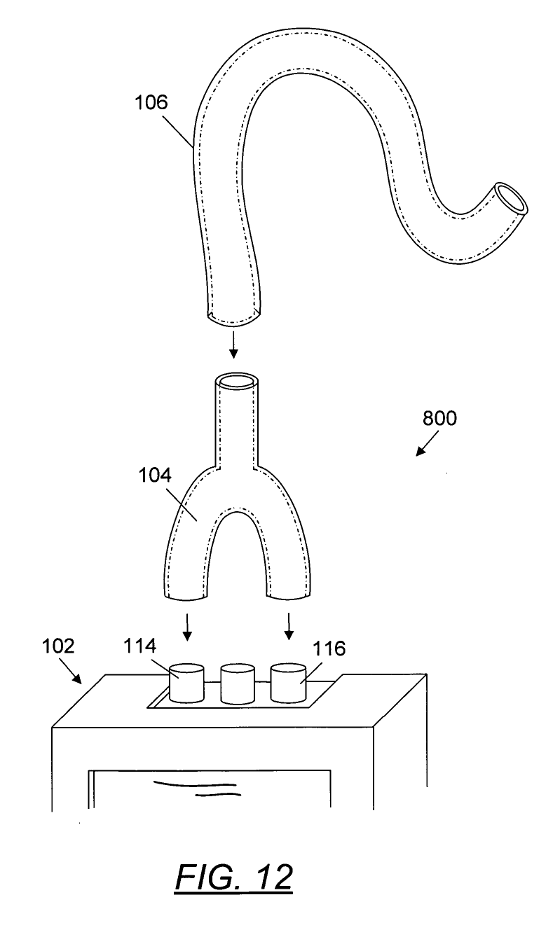

FIG. 12 depicts a detailed view of a portion of the housing and the oral interface depicted in FIG. 3 and how such parts are operably connected. Referring to FIG. 12 and the embodiment 800 depicted, the male portion 104 of the oral interface (or the unitary piece in some embodiments) is operably connected to the housing 102 at air inlets 114, 116 by press or snap fitting to form a substantially airtight seal. The male portion 104 fits within the female portion 106 of the oral interface by press or snap fitting to form a substantially airtight seal.

In an embodiment depicted in FIGS. 13A and 13B, the oral interface 900, 950 comprises a plastic tube with a lumen diameter 915, 965 of from about one to about 10 millimeters, more preferably from about four millimeters to about ten millimeters, and most preferably from about 5 to about 7 millimeters. In oral interface 950, the diameter 965 is divided equally between the two bifurcated parts. By way of example, but not limitation, one may use a surgical tube. Such surgical tubes are well known to those skilled in the art. By way of example, one may use a flexible fluid tubing or surgical tubing as disclosed in U.S. Pat. No. 3,972,321 (Upper Lip Mounted retaining Means for Medical-Surgical Tubes) (reference 18 of FIG. 5); U.S. Pat. No. 4,287,891 (Securing Device for Surgical Tubes); U.S. Pat. No. 4,069,826 (Surgical Tube Adapter Clamp) (reference 3 of Figures); U.S. Pat. No. 5,464,025 (Self-Contained Surgical Tubing Management System) (reference 14 of FIG. 1) or produced by the process of U.S. Pat. No. 4,211,741 (Extrusion process for laminated medical-surgical tubing). In some embodiments, one may also use Y-tubing according to the teachings of U.S. Pat. No. 5,945,052 (Unitary “Y” tubing and method for manufacturing same). In yet other embodiments, one may incorporate a “Y juncture” according to the teachings of U.S. Pat. No. 6,585,708 (Flow control system and method for endoscopic surgeries). By way of further example, one may use Y tubing manufactured by Precision Extrusion, Inc. or distributed by Acmicorp (formerly known as Cabot Medical, 2021 Cabot Blvd. West, Langhorne Pa. 19047.)

In one embodiment depicted in FIG. 13A, the tube 900 permits one way airflow 910 through the tube upon inhalation by the user. In another embodiment depicted in FIG. 13B, said tube 950 is bifurcated and permits bi-directional airflow 960, 962. As will be apparent, one portion of such bifurcated tube may be used to transport filtered breathable air 962 upon inhalation by the user while the second portion may be used to transport exhaust air 960 when the user exhales. Such tubes are well known to those skilled in the art. In yet another embodiment, both portions of the bifurcated tube may be used to transport breathable filtered air from the air chamber to the mouth of the user.

In another embodiment (not shown), a mouthpiece is attached to the second end of the oral interface to provide a more comfortable fit for the user. In one embodiment, said mouthpiece further comprises a valve to control directional airflow. In one embodiment, three unidirectional valves are used, two that permit airflow in the direction of inhalation and one that permits airflow in the direction of exhalation. In another embodiment, two unidirectional valves are used, one that permits airflow in the direction of inhalation and one that permits airflow in the direction of exhalation.

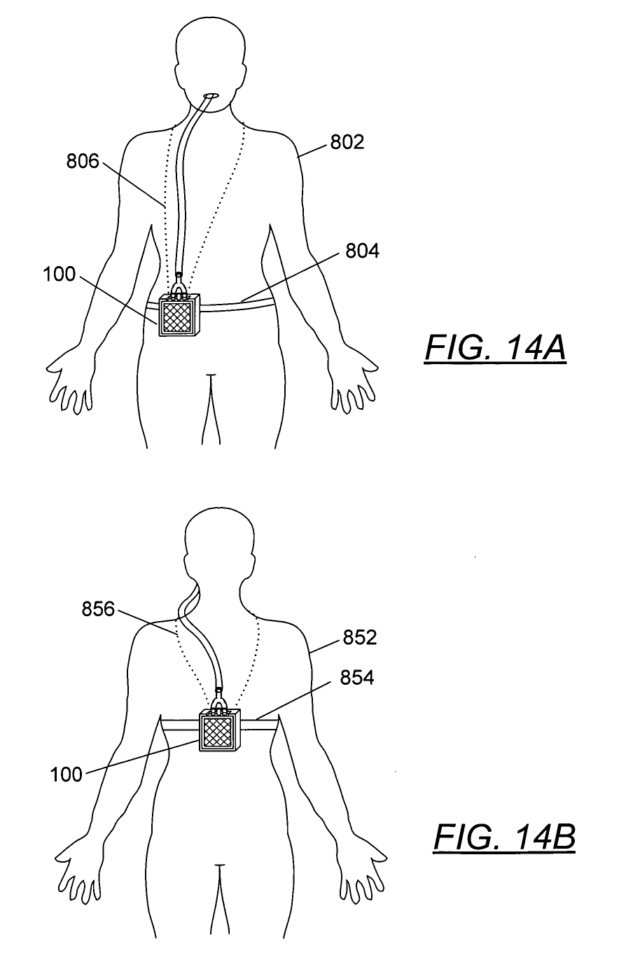

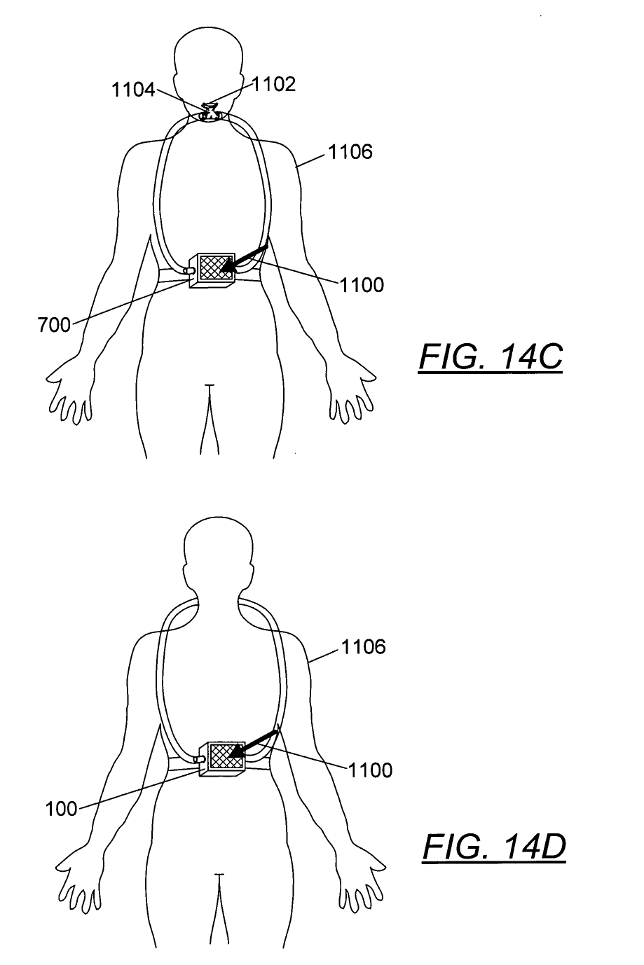

FIG. 14A depicts one demonstration of respirator 100 while in use from a front view of the user 802. FIG. 14B depicts one demonstration of respirator 100 while in use from a rear view of the user 852. Optionally, as depicted in FIGS. 14A and 14B, the portable device 100 may be worn by the user 802, 852. By way of illustration, but not limitation, the device 100 may be secured to the clothing or belt of the user 802, 852. The device 100 may be secured around torso by a belt or strap 804, 854 as depicted in FIGS. 14A and 14B. Securing may also be accomplished by a clip, pins, Velcro, fastener strap or the like. Alternatively, the device 100 may be hung around the neck by a neck strap 806, 856 as depicted by the broken lines in FIGS. 14A and 14B. As will be apparent, the device on a neck strap may be worn on the front of the torso as depicted in FIG. 14A or on the back of the user 852 as depicted in FIG. 14B. Alternatively, the device may be secured to the arm or leg of the user with a strap, belt or other adequate affixing mechanism. Alternatively, the device 100 may be secured to the chest, waist, neck or head of the user in any manner that becomes convenient to the user 802, 852.

Referring to FIG. 15, when using the present invention 100, environmental air is filtered through the filter 806 into the air chamber 814 of the housing, via the oral interface tube 812 into the user's 802 mouth 810 in the direction of 808. Environmental air intake through the filter 806 filters harmful particulates from the air such that breathable air enters the air chamber 814. Such breathable air is inhaled into the user's mouth 810 via the oral interface 812, e.g., surgical tube. In this aspect, the user 802 may exhale through the nose while breathing in through the mouth.

Optionally, in embodiments where a two-way oral interface is used (not shown), the user may use nose clips in combination with the device to prevent environmental air from entering the nostrils. In this aspect, the user will inhale and exhale through the oral interface, discharging exhaust air through the oral interface into the air chamber where it may pass through the filter to the environment or be inhaled by the user or some combination thereof.

FIG. 14C depicts one demonstration of respirator 1000 while in use from a front view of the user. FIG. 14D depicts one demonstration of respirator 1000 while in use from a rear view of the user. Optionally, as depicted in FIGS. 14C and 14D, the portable device may be worn by the user or secured as described above or in any manner convenient to the user. It is preferred that the mouthpiece 1104 be disposed in the user's 1106 mouth 1102. In one embodiment depicted in FIG. 14D, the device 1000 is worn on the back of the user 1106.

Referring to FIGS. 14C and 14D, when using the present invention, environmental air is filtered through the filter 1108 into the air chamber of the housing, through the oral interface tube into the user's 1106 mouth 1102 in the direction of 1100. Environmental air intake through the filter 1108 filters harmful particulates from the air such that breathable air enters the air chamber. Such breathable air is inhaled into the user's 1106 mouth 1102 via the oral interface, e.g., surgical tube. In this aspect, the user may exhale through the nose while breathing in through the mouth.

Optionally, the user may use nose clips in combination with the device to prevent environmental air from entering the nostrils. In this aspect, the user will inhale and exhale through the oral interface, discharging exhaust air through the oral interface into the air chamber where it may pass through the filter to the environment or be inhaled by the user or some combination thereof.

I claim:

1. An oral respirator comprising
a. a filter removably disposed within said oral respirator, wherein said filter is a surgical mask;
b. a housing for receiving and confining said filter comprising
i. an air chamber,
ii. a first air inlet,
iii. a second air inlet, and
iv. a securing assembly for said filter;
c. an oral interface comprising
i. a tubular member operatively connected to said housing, wherein said tubular member comprises
i. a first opening,
ii. a second opening, and
iii. a third opening,
ii. a first valve operatively connected to said tubular member,
iii. a second valve operatively connected to said tubular member,
iv. wherein said oral interface enables a user to breathe through said oral respirator; and
d. wherein said oral respirator filters particulates from breathed air.

2. The oral respirator as recited in claim 1 wherein said first valve and said second valve comprise a unidirectional respiratory valve for allowing the flow of air from said air chamber to an oral cavity of the user when said user inhales through said oral interface.

3. The oral respirator as recited in claim 1 wherein said first valve and said second valve comprise a bidirectional respiratory valve for allowing the flow of air from said air chamber to an oral cavity of the user when said user inhales through said oral interface and the flow of air from said oral cavity of the user into said air chamber when said user exhales through said oral interface.

4. The oral respirator as recited in claim 1 wherein said third opening comprises a mouthpiece.

5. The oral respirator as recited in claim 4 wherein said oral interface further comprises a third valve.

6. The oral respirator as recited in claim 4 wherein said mouthpiece comprises a fourth opening and third valve, wherein said third valve is operably connected to said fourth opening and said third valve is in fluid flow communication with said third opening.

7. The oral respirator as recited in claim 1 wherein said tubular member comprises surgical tubing.

8. The oral respirator as recited in claim 1 wherein said air chamber comprises a volume of from about 5 to about 100 cubic inches.

9. The oral respirator as recited in claim 1 wherein said first air inlet is fluid flow communication with said oral interface.

10. The oral respirator as recited in claim 1 wherein said second air inlet is fluid flow communication with said oral interface.

11. The oral respirator as recited in claim 1 wherein said housing further comprises a plurality of air inlets.

12. The oral respirator as recited in claim 1 wherein said housing comprises a plastic.

13. The oral respirator as recited in claim 1 wherein said housing comprises a polycarbonate composition.

14. The oral respirator as recited in claim 1 wherein said housing further comprises a first portion and a second portion, wherein said first portion comprises an opening of from about 5 to about 20 square inches.

15. The oral respirator as recited in claim 1 wherein said securing assembly for said filter comprises a prong and hole assembly.

16. The oral respirator as recited in claim 1 wherein said securing assembly for said filter comprises a rib and groove assembly.

17. The oral respirator as recited in claim 1 wherein said oral interface further comprises a fitting operatively connected to said housing and said tubular member.

18. The oral respirator as recited in claim 1 wherein said oral interface further comprises a protective shield.

19. An oral respirator comprising
a. a filter removably disposed within said oral respirator, wherein said filter is a surgical mask;
c. a housing for receiving and confining said filter comprising
v. an air chamber,
vi. an air inlet, and
vii. a securing assembly for said filter;
c. an oral interface comprising
v. a tubular member operatively connected to said housing, wherein said tubular member comprises
i. a first opening, and
ii. a second opening,
vi. a first valve operatively connected to said tubular member,
vii. wherein said oral interface enables a user to breathe through said oral respirator; and
d. wherein said oral respirator filters particulates from breathed air.

20. A process for filtering particulates from breathed air comprising
a. a step of disposing and confining a surgical mask within a housing of an oral respirator wherein said housing comprises an air chamber, a first air inlet, a second air inlet, and a securing assembly for said filter;
b. a step of inserting an oral interface into an oral cavity, wherein said oral interface comprises a tubular member operatively connected to said housing, wherein said tubular member comprises a first opening, a second opening, and a third opening, a first valve operatively connected to said tubular member, a second valve operatively connected to said tubular member, and wherein said oral interface enables a user to breathe through said oral respirator; and
c. a step of inhaling via said oral interface wherein environmental air passes through said surgical mask into said air chamber thereby becoming filtered breathable air, and wherein said filtered breathable air passes into said oral interface via said first air inlet and said second air inlet and into said oral cavity.
